(19) United States
(12) Patent Application Publication
(10) Pub. No.: US 2010/0239523 A1
(43) Pub. Date: Sep. 23, 2010

(54) TLR MODULATORS AND METHODS FOR USING THE SAME

(75) Inventors: Linda May Rothblum Watkins, Boulder, CO (US); Mark Rowland Hutchinson, Glenalta (AU)

Correspondence Address: Don D. Cha, 547 Buena Vista Road, Golden, CO, 80401, US

(73) Assignee: The Regents of the University of Colorado, Denver, CO (US)

(21) Appl. No.: 12/740,690

(22) PCT Filed: Oct. 30, 2008
(86) PCT No.: PCT/US08/81862

§ 371 Date: Apr. 29, 2010

Related U.S. Application Data
(60) Provisional application No. 60/983,924, filed on Oct. 30, 2007.

(51) Int. Cl. A61K 38/20; A61P 3/10; A61P 1/00; A61P 9/00; A61P 25/04; A61P 37/00; A61P 25/28; A61P 15/04; A61P 29/00; A61P 25/06; A61P 25/08; A61P 25/36; A61K 31/485; A61K 31/437; A61K 31/138; A61K 31/366; A61K 31/167; A61K 31/522; A61K 31/19; A61K 31/46; A61K 31/4168; A61K 31/65

(52) U.S. Cl. 424/85.2; 514/282; 514/300; 514/648; 514/456; 514/626; 514/263.36; 514/578; 514/291; 514/553; 514/401; 514/152; 514/154

(57) ABSTRACT

The invention provides Toll-like receptor (TLR) modulators, compositions comprising the same, and methods for using the same.

### CROSS-REFERENCE TO RELATED APPLICATIONS

This application claims the priority benefit of U.S. Provisional Application No. 60/983,924, filed Oct. 30, 2007, which is incorporated herein by reference in its entirety.

### STATEMENT REGARDING FEDERALLY FUNDED RESEARCH

The U.S. Government has a paid-up license in this invention and the right in limited circumstances to require the patent owner to license others on reasonable terms as provided for by the terms of Grant Nos. DA015642, DA017670, and DE017782 awarded by the National Institutes of Health.

### FIELD OF THE INVENTION

The invention relates to Toll-like receptor (TLR) modulators, compositions comprising the same, and methods for using the same.

### BACKGROUND OF THE INVENTION

The pharmacology and treatment of pain has a very long and tumultuous history. Since the infancy of the use of opium poppy extracts to treat pain around 3500 BC, the search for treatments that provide effective relief from acute and chronic pain has continued to grow at an extraordinary rate. Today, pain still remains a significant public health issue with two-thirds of patients achieving little to no pain relief from the myriad of currently available pharmacotherapies and dosing regimens. The use of opioid pharmacotherapies produces several rewarding and reinforcing side effects, which result in the drugs' diversion to abuse settings. Unfortunately, a significant side effect in attempting to improve patients' quality of life is that some become dependent to the treatments that were prescribed to help them. In recent years the misuse of opioids has risen drastically, leaving doctors and patients hesitant to treat pain to the fullest extent.

Therefore, there is a continuing need for compounds, compositions, and methods for treating pain.

### SUMMARY OF THE INVENTION

Some aspects of the invention provide methods for using a toll like receptor (TLR) modulator. In some embodiments, TLR modulators are TLR antagonists.

In other aspects of the invention, methods are provided for modulating TLR comprising contacting a cell expressing a TLR with an effective amount of a compound that modulates TLR-2, TLR-4, other TLR or a combination thereof. In some embodiments, the compound antagonizes or inhibits TLR-2, TLR-4, other TLR or a combination thereof. When the compound is a chiral compound, in some instances the compound is enantiomerically enriched. Within these instances, in some cases the compound is enantiomerically enriched (+)-isomer. In some cases, the compound is enantiomerically enriched (−)-isomer. In other cases, the compound is a mixture of the (+)- and the (−)-isomers, such as a racemic mixture. Yet in other embodiments, methods of the invention modulate TLR mediated signaling that is generated in response to a TLR ligand.

Other aspects of the invention provide methods for treating a subject for a clinical condition associated with TLR activation. The methods generally comprise administering to the subject a compound that modulates TLR-2, TLR-4, other TLR that recognizes endogenous danger signals or opioids or non-opioid analgesics, or a combination thereof. Often the compound antagonizes or inhibits TLR-2, TLR-4, other TLR, or a combination thereof.

Still other aspects of the invention provide methods for treating a subject for a clinical condition associated with TLR mediated glial activation. Methods typically comprise administering to the subject a compound that modulates, often antagonizes or inhibits, a TLR. In some embodiments, clinical conditions include chronic pain conditions (including but not limited to neuropathic pain), TMJ disease, spinal cord injury pain, radiculopathy, arthritis of various etiologies, visceral pain (including but not limited to colitis, nephritis, pancreatitis, and irritable bowel syndrome), cancer pain, vulvadynia, spinal stenosis, fibromyalgia, post-stroke pain and other chronic pains of central nervous system origin, multiple sclerosis pain, craniofacial pain syndromes of various etiologies, and the like; acute, repetitive, and chronic opioid and non-opioid analgesia, or a unwanted opioid side-effect, or a combination thereof. The term “unwanted opioid side-effect” refers to opioid effects other than analgesia.

The clinical conditions that can be treated with various methods of the invention also include, but not be limited to, gastrointestinal pathologies (e.g., colitis, inflammatory bowel disease, Crohn's disease, irritable bowel disease, and celiac disease), cardiovascular disease (e.g., inflammatory heart disease, vascular inflammation, myocardial ischemia/reperfusion injury, and atherosclerosis), diabetes [e.g., diabetes/insulin resistance, (killing of islet cells)], immune related conditions (e.g., allergy, asthma, eczema, auto-immune disorders including arthritis, lupus and glomerulonephritis), systemic pathologies (e.g., primary or secondary sepsis, transplant organ rejection, and liver toxicity), neurodegeneration (e.g., neurodegenerative disorders generally, including Alzheimer's, Parkinson's, dementia, Multiple Sclerosis, Huntington's disease, Amyotrophic lateral sclerosis, and aging), and other physiological function (e.g., induction of labor, fever, seizures, epilepsy, and epileptogenesis).

In other embodiments, the clinical condition comprises chronic pain, acute, repetitive or chronic opioid or non-opioid analgesia, or a unwanted opioid side-effect, gastrointestinal pathologies, cardiovascular disease, diabetes, immune related conditions, systemic pathologies, neurodegeneration, induction of labor, fever, seizures, epilepsy, epileptogenesis, or a combination thereof. Within these embodiments, in some instances the unwanted opioid side-effect comprises opioid dependence, opioid reward, opioid induced respiratory depression, opioid induced ataxia, opioid induced hyperalgesia, opioid induced allodynia or hyperalgesia, opioid induced gastrointestinal disorders, opioid dysphoria, or a combination thereof.

Still in other embodiments, the compound antagonizes or inhibits TLR-2, TLR-4, other TLR that is activated by endogenous danger signals or opioids, or a combination thereof. In other embodiments, the compound antagonizes or inhibits TLR-2, TLR-4, other TLR or a combination thereof.

Other aspects of the invention provide methods for treating neuropathic pain or other painful and non-painful conditions in a subject, said method comprising administering to the subject in need of such a treatment a compound that modulates, often antagonizes or inhibits, a TLR.

Some aspects of the invention provide methods for treating a clinical condition associated with agonism of TLR.

Other aspects of the invention provide a method for treating a subject for a clinical condition associated with TLR activation by administering to the subject a compound that modulates TLR-2, TLR-4, or a combination thereof. In some embodiments, the method also includes administering an enantiomerically enriched (−)-opioid. In such cases, sometimes the enantiomerically enriched (−)-opioid and the compound that modulates TLR-2, TLR-4, or a combination thereof are co-administered. Exemplary clinical conditions that can be treated with methods of the invention include, but are not limited to, chronic pain, acute opioid analgesia, or a unwanted opioid side-effect, gastrointestinal pathologies, cardiovascular disease, diabetes, immune related conditions, systemic pathologies, neurodegeneration, induction of labor, fever, seizures, epilepsy, epileptogenesis, or a combination thereof. In some instances, the clinical condition associated with TLR activation comprises chronic pain, acute opioid analgesia, or an unwanted opioid side-effect, or a combination thereof. In some cases, the unwanted opioid side-effect comprises opioid dependence, opioid reward, opioid induced respiratory depression, opioid induced ataxia, opioid induced hyperalgesia, opioid induced allodynia or hyperalgesia, opioid induced gastrointestinal disorders, narcotic bowel syndrome, opioid dysphoria, or a combination thereof. In some embodiments, the enantiomerically enriched (−)-opioid comprises morphine or a derivative or an analog thereof. Still in other embodiments, the compound that modulates TLR-2, TLR-4, or a combination thereof comprises a compound of FIG. 3 or a derivative or an analog thereof. Often the compound is a TLR antagonist. In some embodiments, the clinical condition associated with TLR activation comprises a clinical condition associated with activation of glial.

Other aspects of the invention comprise a method for treating neuropathic pain in a subject. The method generally comprises administering to the subject in need of such a treatment a TLR antagonist compound. In some embodiments, the method further comprises administering an enantiomerically enriched (−)-opioid. In some instances, enantiomerically enriched (−)-opioid and the TLR antagonist compound are co-administered. In other embodiments, the enantiomerically enriched (−)-opioid is morphine or a derivative or an analog thereof. In other embodiments, the method for treating neuropathic pain in a subject comprises administering to the subject a TLR antagonist compound in the absence of (−)-opioids, as TLR antagonists can be stand alone treatments for neuropathic pain as well adjunct therapies.

Still other aspects of the invention provide an analgesic composition comprising an admixture of an enantiomerically enriched analgesic (−)-opioid and a TLR antagonist. In some embodiments, the analgesic (−)-opioid comprises morphine or a derivative or an analog thereof. In other embodiments, TLR antagonist antagonizes TLR-2, TLR-4, or a combination thereof. In some particular embodiments, the TLR antagonist comprises a compound of FIG. 3 or a derivative or an analog thereof.

### BRIEF DESCRIPTION OF THE DRAWINGS

FIG. 1 is a schematic illustration of neuropathic pain highlighting theoretical points (A through E) where some of the pharmacological targets can be designed to treat neuropathic pain to which glia contribute.

FIG. 2 is a graph showing that chronic constriction injury (CCI)-induced allodynia is reversed by acute blockade of TLR4, demonstrating that TLR antagonists can be stand alone therapies for neuropathic pain.

FIG. 3 is a table of in vitro TLR antagonism data for various compounds.

FIG. 4 is a graph showing reversal of chronic constriction injury (CCI)-induced neuropathic pain by acute intrathecal delivery of a TLR-4 antagonist, mutant lipopolysaccharide (LPS), and LPS-RS.

FIG. 5 is a graph showing inhibition of TIRAP signaling potentiates intrathecal morphine analgesia.

FIG. 6A is a graph showing potentiation of morphine analgesia by co-administration of LPS antagonist.

FIG. 6B is a graph showing potentiation of morphine analgesia by co-administration with quercetin.

### DETAILED DESCRIPTION OF THE INVENTION

### Definitions

“Antagonist” refers to a compound or a composition that attenuates the effect of an agonist. The antagonist can bind reversibly or irreversibly to a region of the receptor in common with an agonist. Antagonist can also bind at a different site on the receptor or an associated ion channel. Moreover, the term “antagonist” also includes functional antagonist or physiological antagonist. Functional antagonist refers to a compound and/or compositions that reverses the effects of an agonist rather than acting at the same receptor, i.e., functional antagonist causes a response in the tissue or animal which opposes the action of an agonist. Examples include agents which have opposing effects on an intracellular second messenger, or, in an animal, on blood pressure. A functional antagonist can sometimes produce responses which closely mimic those of the pharmacological kind.

“Chronic pain” refers to pain that persists longer than the temporal course of natural healing, associated with a particular type of injury or disease process.

“Nociceptive pain” refers to pain associated with the nerves which sense and respond to parts of the body which suffer from damage. Nociceptive pain is generally caused by an injury or disease outside the nervous system. It is often an on-going dull ache or pressure, rather than the sharpter, trauma-like pain more characteristic of neuropathic pain. They signal tissue irritation, impending injury, or actual injury. When activated, they transmit pain signals (via the peripheral nerves as well as the spinal cord) to the brain. The pain is typically well localized, constant, and often with an aching or throbbing quality. Visceral pain is the subtype of nociceptive pain that involves the internal organs. It tends to be episodic and poorly localized. Nociceptive pain is usually time limited, e.g., when the tissue damage heals, the pain typically resolves. (Arthritis is a notable exception in that it is not time limited.) Typically, nociceptive pain tends to respond well to treatment with opioids. Exemplary nociceptive pains include sprains, bone fractures, burns, bumps, bruises, inflammation (from an infection or arthritic disorder), obstructions, and myofascial pain (which may indicate abnormal muscle stresses).

“Pharmaceutically acceptable excipient” refers to an excipient that is useful in preparing a pharmaceutical composition that is generally safe, non-toxic and neither biologically nor otherwise undesirable, and includes excipient that is acceptable for veterinary use as well as human pharmaceutical use.

“Pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The terms “pro-drug” and “prodrug” are used interchangeably herein and refer to any compound which releases an active parent drug according to those disclosed herein in vivo when such prodrug is administered to a mammalian subject. Prodrugs of a compound that is disclosed herein are prepared by modifying one or more functional group(s) present in the compound in such a way that the modification(s) may be cleaved in vivo to release the parent compound. Prodrugs include compounds disclosed herein wherein a hydroxy, amino, or sulfhydryl group in the compound is bonded to any group that may be cleaved in vivo to regenerate the free hydroxyl, amino, or sulfhydryl group, respectively. Examples of prodrugs include, but are not limited to, esters (e.g., acetate, formate, and benzoate derivatives), carbamates (e.g., N,N-dimethylaminocarbonyl) of hydroxy functional groups in the compounds disclosed herein, and the like.

“A therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Treating” or “treatment” of a disease includes: (1) preventing the disease, i.e., causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

“Enantiomeric excess” refers to the difference between the amount of enantiomers. The percentage of enantiomeric excess (% ee) can be calculated by subtracting the percentage of one enantiomer from the percentage of the other enantiomer. For example, if the % ee of (R)-enantiomer is 99% and % ee of (S)-enantiomer is 1%, the % ee of (R)-isomer is 99%-1% or 98%.

The term “a derivative or an analog thereof” refers to those compounds that are derived from or having a similar core structure and retain all of the biological activity of the compound to which they are referred to. The term “all of the biological activity” refers to biological activities referred to herein when discussing the compound, e.g., TLR antagonistic property, etc.

### Overview

Owing to the pain transmission capacity, neurons have been the primary intentional target of all pharmacotherapies developed to date. Generally, it is believed that opioids modulate pain almost exclusively by acting at neuronal opioid receptors and that opioid antagonists likewise exert their effects almost exclusively on neurons. Furthermore, it is conventionally believed that detrimental (e.g., tolerance, hyperalgesia, dependence, reward, respiratory depression, ataxia, sedation, etc.) and beneficial (e.g., analgesia, cough suppressant, etc.) actions of opioids are mediated via very similar and potentially inseparable mechanisms, reliant on neuronal opioid receptors.

However, the present inventors have discovered that the immunocompetent cells of the central nervous system (glia), their receptors, and their secreted signaling factors are involved in pain processing and opioid pharmacodynamics. In particular, glia have been shown to have a role in initiating and maintaining increased nociception in response to peripheral nerve injury. Recently, it has been suggested that glia can also modulate the analgesic actions of chronically administered opioids. Accordingly, some aspects of the invention provide pharmacological targeting (e.g., modulation) of glia to modulate (e.g., reduce or eliminate) pain and enhanced efficacy of opioids.

The present inventors also have shown that opioids cause direct glial activation in a non-classical opioid receptor fashion, via opioid-induced activation of a class of pattern recognition receptors termed Toll-like Receptors (TLRs). TLRs are significant mediators of neuropathic pain, opioid tolerance, opioid dependence, opioid reward and other negative side effects. Thus, in some instances antagonizing TLRs reverses neuropathic pain, and potentiates opioid and non-opioid analgesia. Also disclosed herein are the beneficial (e.g., classical neuronal opioid receptor mediated analgesia) and detrimental (e.g., glially mediated side effects) actions of analgesic compounds, such as opioids, and methods for modulating such.

As disclosed herein, glial activation also contributes significantly to neuropathic pain and to the development of opioid tolerance, opioid dependence and opioid reward. Thus, attenuation of glial activation alleviates neuropathic pain and reduces the development of opioid tolerance, dependence and reward. Without being bound by any theory, it is believed that opioid-induced glial activation occurs via a non-opioid receptor due to non-stereoselective agonist activity. Accordingly, some aspect of the invention relates to attenuating glial activation by antagonizing or blocking TLR (e.g., TLR2, TLR4, other TLR that can bind to either opioid analgesics, non-opioid analgesics or endogenous danger signals known to be TLR agonists, or a combination thereof) or generally reducing glial activation. Reduction of glial activation reduces exaggerated pain states, enhances opioid analgesia, and reduces the risk of developing opioid tolerance, dependence and reward.

Some of the other clinical conditions associated with TLR include, but are not limited to, gastrointestinal pathologies (e.g., colitis, inflammatory bowel disease, Crohn's disease, irritable bowel disease, and celiac disease), cardiovascular disease (e.g., inflammatory heart disease, vascular inflammation, myocardial ischemia/reperfusion injury, and atherosclerosis), diabetes [e.g., diabetes/insulin resistance, (killing of islet cells)], immune related conditions (e.g., allergy, asthma, eczema, auto-immune disorders including arthritis, lupus and glomerulonephritis), systemic pathologies (e.g., primary or secondary sepsis, transplant organ rejection, and liver toxicity), neurodegeneration (e.g., neurodegenerative disorders generally, including Alzheimer's, Parkinson's, dementia, Multiple Sclerosis, Huntington's disease, Amyotrophic lateral sclerosis, and aging), and other physiological function (e.g., induction of labor, fever, seizures, epilepsy, and epileptogenesis). Accordingly, some aspects of the invention provide methods for treating a clinical condition associated with agonism of TLR.

Conventionally, glia (astrocytes and microglia) were viewed as structural supports for neurons and important for maintaining central nervous system (CNS) homeostasis. Glia were long overlooked in pain research due to their lack of axons and their yet-to-be-discovered roles in cell-to-cell communication. The roles of CNS glia in providing immune surveillance, clearance of debris, and regulation of ionic and chemical composition of the extracellular space in the survival of the host are well known. However, a possible involvement of glia under varying pain states has only recently been investigated. One possible indication for a potential role of glia in pain regulation is an associative link between astrocyte activation and neuropathic pain, for example, drugs that blocked neuropathic pain also decreased glial activation.

Upon activation, the functions of microglia and astrocytes change in that they begin producing and releasing a variety of neuroexcitatory substances including traditional nociceptive modulators, such as reactive oxygen species, nitric oxide, prostaglandins, excitatory amino acids, growth factors, and proinflammatory cytokines, which was recently recognized. Principal among proinflammatory cytokines are interleukin (IL)-1, IL-6 and tumor necrosis factor-α. Without being bound by any theory, it is believed that spinal cord glia are one of the principal producers of these proinflammatory cytokines in the central nervous system. In fact, spinal glial activation and subsequent release of proinflammatory mediators are believed to be involved in initiating and maintaining diverse enhanced pain states including neuropathic pain.

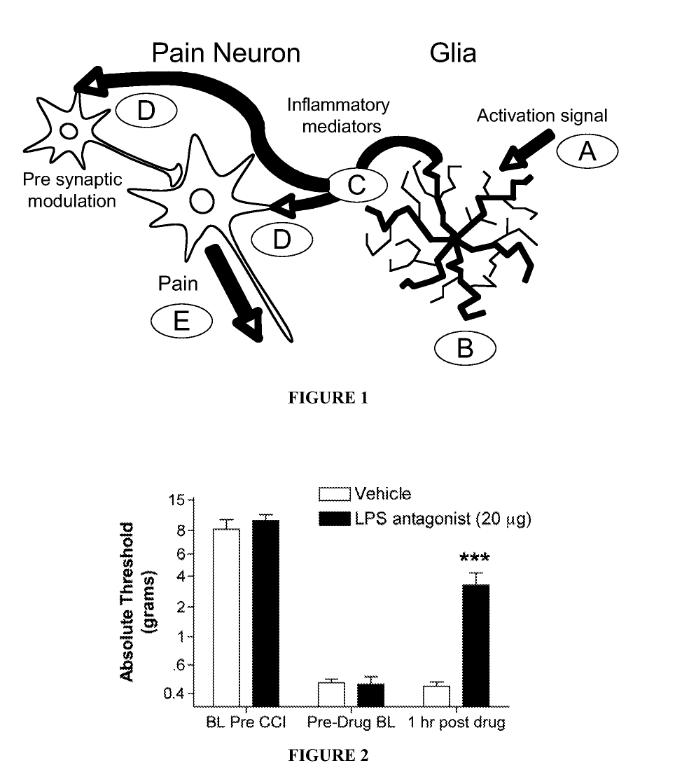

As shown in FIG. 1, there are numerous points along glial regulation of neuropathic pain where glia can be targeted to treat neuropathic pain. Traditional pain therapies have typically targeted transmission of the pain signal via neurons (step E; FIG. 1) with limited success. As can be seen in this schematic pathway, merely treating the neuronal component of the pathology leaves the glial component unabated, still attempting to communicate to neurons to propagate pain signals. It is possible glia are activating neurons via different pathways/intracellular signaling cascades than modulated by drugs targeting neurons. Perhaps this explanation may elucidate the unfortunate lack of generalized success of current pain therapies.

One of the initial steps in the neuropathic pain pathway is believed to be activation of glia (Step A in FIG. 1). A variety of glial activation signals have been identified. Signal(s) that initiates glial activation can vary depending on the insult delivered. Several mediators of glial activation are well characterized including neuronally-released fractalkine and traditional neuronal nociceptive modulators and transmitters, such as reactive oxygen species, nitric oxide, prostaglandins, excitatory amino acids, substance P, ATP, growth factors, and proinflammatory cytokines. In the majority of these cases, known receptor-mediated events have been characterized.

As can be seen in FIG. 1, a variety of points in neuropathic pain (A through E) can be targeted to treat neuropathic pain to which glia contribute. An activation signal or series of activation signals are required to activate glia (Step A in FIG. 1). Activation of glia is often mediated via cell surface receptors that can be antagonized. The term “glial activation” refers to the state in which glia release proinflammatory mediators. This state (i.e., glial activation, Step B in FIG. 1) can be modulated or attenuated thereby inhibiting various cellular events that block glial activation or its downstream consequences. An anti-inflammatory environment can also be produced which increases the threshold that an activation signal has to overcome to activate the cells.

Immune inflammatory mediators such as proinflammatory cytokines can be neutralized prior to reaching their intended receptor target (pre and/or post synaptic) by using soluble receptors (which exist endogenously), neutralizing antibodies, or compounds that decrease maturation of cytokines into their active form or increase the rate of cytokine degradation (Step C in FIG. 1). The action of many glial inflammatory mediators on neurons (pre and/or post synaptic) can also be antagonized at neuronal receptor sites (Step D in FIG. 1). There are myriads of currently employed neuronally targeted therapies that decrease the neuronal signaling of pain signals (pre and/or post synaptic, Step E in FIG. 1).

Some aspects of the invention relate to modulating initiator and mediator of neuropathic pain that involve signals relayed by Toll-like Receptors (TLRs), such as TLR2, TLR4, other TLR that recognizes endogenous danger signals or opioids or non-opioids, or a combination thereof. TLRs are a family of approximately 10 single transmembrane receptors that recognize a diverse range of moieties or “patterns” on exogenous (e.g., lipopolysaccharide [LPS] of gram-negative bacteria such as E. coli and Salmonella) and endogenous (e.g., heat shock proteins and cell membrane components released from damaged cells) substances that are considered to be danger signals and hence warrant activation of the innate immune system aimed at defending the survival of the host. TLR4 has been extensively characterized, as it is the TLR that recognizes LPS. Binding of agonists to TLRs activate downstream intracellular signaling pathways (similar to IL-1 binding to its cognate receptor) resulting in a proinflammatory signal.

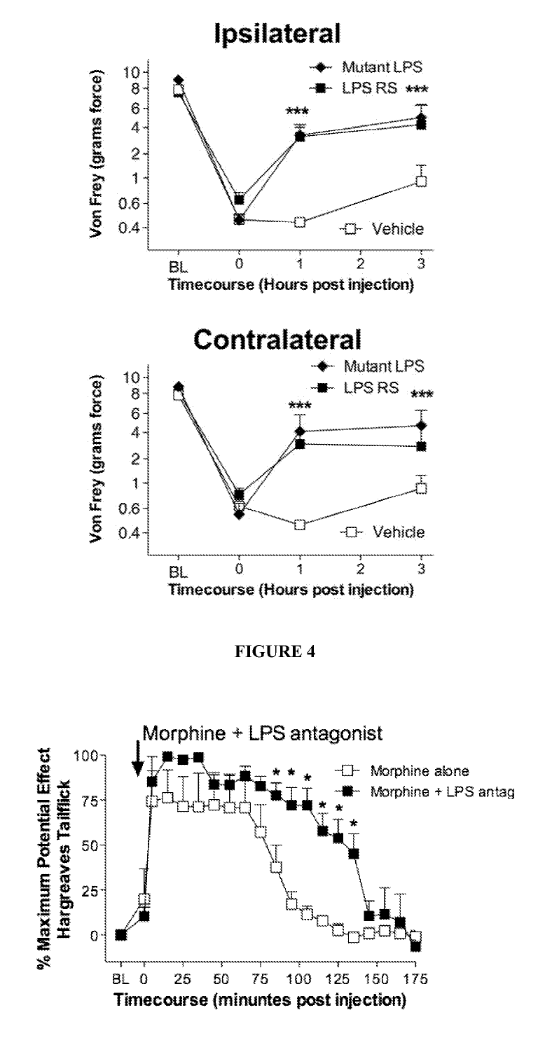

Some aspects of the invention modulate TLR2, TLR4, other TLR that can bind to either opioid analgesics, non-opioid analgesics or endogenous danger signals known to be TLR agonists, or a combination thereof. As disclosed herein, a wide variety of chemically diverse compounds can modulate TLR2, TLR4, other TLR as above, or a combination thereof. Without being bound by any theory, using TLR2 and TLR4 as exemplars, TLR2 and TLR4 are believed to be some (but not all) of the key TLRs for recognizing and responding to endogenous danger signals that are released by damaged, dying and dead neurons and other cells (host DNA and RNA, heat shock proteins, cell membrane components, etc) and more general aspects of tissue injury (plasma proteins, extracellular matrix degradation products, etc). The present inventors have shown that acute intrathecal administration of a selective TLR4 antagonist in normal rats suppresses well-established neuropathic pain induced by chronic constriction injury (see FIGS. 2 and 4).

FIG. 2 is a bar graph showing that when adult rats that were administered intrathecally with 20 μg of TLR4 antagonist (a mutant LPS from msbB mutant E. coli that binds to but does not activate TLR4; Invivogen, San Diego, Calif., USA), it acutely reverses allodynia induced by chronic constriction injury of the sciatic nerve (n=6 per group). This TLR4 antagonist is a mutant form of LPS which lacks the myristoyl fatty acid moiety of the lipid A and displays 1,000 to 10,000 fold reduction in activation of NF-κB. In FIG. 2, BL (baseline) pre CCI Von Frey testing of thresholds occurred the day of CCI surgery of the left sciatic nerve. Allodynia developed across the following 10 days with maximal allodynia achieved and maintained from this point for a further 21 days. Pre-drug baseline (BL) thresholds were assessed the morning of the test day. Acute intrathecal delivery via the lumbar approach of vehicle and drug was conducted under brief isoflurane anesthesia. One hour post drug Von Frey testing was conducted. All testing was conducted blinded to treatment group by two separate experimenters.

FIG. 4 is a graph showing reversal of chronic constriction injury (CCI)-induced neuropathic pain by acute intrathecal delivery of a TLR-4 antagonist, mutant lipopolysaccharide (LPS), and LPS-RS. After baseline (BL) testing, rats received CCI of one sciatic nerve at the mid-thigh level. After pre-drug testing (0 h) 14 days later to confirm the development of bilateral CCI-induced mechanical allodynia, rats were intrathecally given either 20 μg of mutant LPS (filled diamonds), 20 μg of LPS-RS (filled squares), or an equal volume of vehicle (open squares). Behavioral responses recorded 1 and 3 h later revealed reliable attenuation of both ipsilateral (A) and contralateral (B) mechanical allodynia by this TLR4 antagonist. In these graphs, P<0.001 as compared to vehicle (saline) controls.

These data demonstrate ongoing stimulation of TLRs, which is likely caused by injury-induced endogenous danger signals. Peripheral nerve injury leads to protracted expression of heat shock proteins in proximal axons of damaged sensory neurons and degradation of presynaptic terminals. Nerve degeneration in the central nervous system occurs remarkably slowly, taking months to years. Therefore, it is clear that endogenous danger signals created as a result of nerve injury could produce perseverative activation of at least TLR2 and TLR4 and, thereby, a perseverative drive for maintaining neuropathic pain. A parallel activation of at least TLR2 and TLR4 would be anticipated to occur in, and be causal to, spinal cord injury pain, post-stroke pain, multiple sclerosis pain and other pains of central nervous system origin. Accordingly, modulation of glial activation (Step A of FIG. 1) can be used to treat neuropathic pain or like pains.

Some aspects of the invention provide compounds and compositions that can modulate (e.g., antagonize) TLRs for neuropathic pain control. Given that TLR2, TLR4, and other TLRs can signal the presence of endogenous danger signals, opioids or non-opioids, some embodiments of the invention provide compounds and compositions that modulates TLR2, TLR4, other TLRs, or a combination thereof. In some embodiments, compounds and compositions of the invention are permeable to the blood-brain barrier.

The classical opioid receptor binds (−)-isomers of opioids selectively. The present inventors have found that a wide variety of compounds are capable of blocking LPS-induced activation of TLR4. Using a TLR4 stably transfected cell line (Invivogen) with a stable co-transfection of an NF-κB reporter gene (secreted embryonic alkaline phosphatase; SEAP) the present inventors have found a significant non-competitive antagonism of LPS activity at TLR4. Other TLR4 antagonists are competitive antagonists of LPS activity at TLR4. See the Examples section.

Compounds of the invention also reverse CCI-induced allodynia following a systemic or central (such as intrathecal) administration. Such results indicate that blood brain barrier permeable small molecules can be used to antagonize TLR4 activity in vivo. In addition, TLR4 antagonism by small molecules can successfully reverse CCI-induced allodynia (Step A of FIG. 1). These data also show a role of TLR4 receptors in neuropathic pain. It is believed that opioid analgesia would be unaffected owing to the lack of opioid activity of the compounds of the invention. Without being bound by any theory, it is believed that compounds of the invention reverse neuropathic pain by non-stereoselectively antagonizing TLR4 receptors.

Compounds of the invention also reverse established allodynia and other neuropathic pain. Without being bound by any theory, it is believed that this activity is achieved via its actions as a TLR4 antagonist.

The mode of glial activation that results in enhanced pain can vary depending on the insult delivered. Thus, an effective treatment for neuropathic pain typically depends on which glial activating signal(s) are responsible for the pain pathway. A broader therapeutic approach is to inhibit or attenuate existing glial activation and/or products released by activated glia (e.g., Step D in FIG. 1). In some instances, compounds of the invention reverse neuropathic pain and return the animal toward normal basal pain responsivity, rather than producing analgesia. Therefore, all of these treatments are anti-allodynic and/or anti-hyperalgesic, leaving basal nociception unaffected.

As discussed previously, the inflammatory and pro-nociceptive mediators released by glia in their activated state are numerous. Therefore, clinically antagonizing or neutralizing each mediator (targeting steps C and/or D of FIG. 1) has its limitations as inhibiting the actions of each of these numerous mediators individually may be too great a task. However, in some instances proinflammatory cytokines appear to be one of the key factors in glial enhancement of pain. In some cases, neutralizing the action of principal proinflammatory cytokines (IL-1, IL-6, tumor necrosis factor-α, for example, Step C in FIG. 1) or antagonizing their receptors (e.g., Step D in FIG. 1) has proven a successful strategy for preventing and reversing neuropathic pain.

It has been observed that there is a similarity between the glial activation observed in response to peripheral neuropathy and the glial activation following chronic opioid exposure. It has also been observed that opioid agonists activate TLR2, TLR4, other TLR, or a combination thereof and compounds of the invention non-stereoselectively block one or more of these receptors.

The present inventors have found that TLRs are responsible for both neuropathic pain and opioid-induced glial activation. Accordingly, some aspects of the invention provide methods for modulating neuropathic pain, opioid-induced glial activation, or a combination thereof by administering a TLR antagonist or a composition comprising the same. In some embodiments, the TLR antagonist does not significantly compromise the pain-suppressive effects of opioids agonists on neurons.

Since the discovery of morphine modulation of T cell function in 1979, a large amount of work has been focused on characterizing the influence that opioid exposure has on the functioning of the immune system in its traditional role of host defense. However, the impact that the activation status of immunocompetent cells has on opioid actions has only been recently studied. While modulation of peripheral immune cells function by opioids is important to understanding host defense, these cells are not as likely as glia to have a profound effect on opioid pharmacodynamics. The immunocompetent cells that mediate effects on opioid analgesia are typically the glia of the dorsal root ganglia, spinal cord and brain. Peripheral immune cells have been implicated in many TLR-mediated clinical diseases, such as Crohn's disease.

A causal link between opioid-induced glial activation and the development of opioid tolerance has recently been recognized. It is believed that following chronic morphine administration, tolerance and morphine-induced hyperalgesia are produced, at least in part, as a consequence of glial activation. One mechanism that has been proposed to account for such effects is via nitric oxide induced p38 MAPK activation, with downstream up regulation of proinflammatory cytokines. Interleukin-1, interleukin-6 and tumor necrosis factor, in turn, oppose morphine analgesia.

It is believed that morphine is acting not only at classical opioid receptors on nociceptive neurons (step E, FIG. 1) but also as a glial activation signal (step A, FIG. 1) producing the same, or at least a similar cascade of events that results in increased nociception. The sum of morphine's neuronal anti-nociceptive activity and its pro-nociceptive glial activation results in a net reduction in analgesia. Moreover, glial activation increases with prolonged opioid treatment and results in an increasing analgesic tolerance. Furthermore, opioid-induced glial activation contributes significantly to the atypical allodynia and hyperalgesia that results from chronic opioid administration. The present inventors have found that IL-1, as well as other proinflammatory cytokines and chemokines, opposes morphine analgesia within minutes after either systemic or intrathecal administration.

The present inventors have observed similarity between neuropathy- and opioid-induced glial activations by using agents that reverse nerve injury-induced allodynia so as to define whether these same agents modulate morphine analgesia as well. The present inventors have discovered that agents that oppose neuropathic pain either by suppressing glial activation or by neutralizing or antagonizing proinflammatory glial products also oppose glial attenuation of both acute and chronic morphine analgesia. The efficacy of morphine can be potentiated by targeting opioid-induced glial activation (step B, FIG. 1) or by neutralizing (step C, FIG. 1) or antagonizing (step D, FIG. 1) the action proinflammatory cytokines.

It is believed that the activation of glia is not mediated via a classical “neuronal-like” opioid receptor. Using TLR4 antagonists that possess no classical opioid receptor activity, the present inventors have discovered the involvement of this non-classical opioid receptor in glial activation, which causes significant glial activation, allodynia and hyperalgesia, as well as upregulation of proinflammatory cytokine mRNA, protein and release. Glia do express classical opioid receptors. However, it is believed that the immunomodulation resulting from opioid exposure is not mediated by these classical opioid receptors.

Some aspects of the invention provide methods for using TLR antagonists to potentiate (−)-opioid (e.g., morphine) analgesia, for example, by blocking (−)-opioid induced glial activation and consequent increase in anti-analgesic proinflammatory cytokines. In some embodiments, TLR antagonists significantly potentiated both acute and chronic (−)-opioid analgesia.

Without being bound by any theory, it is believed that (−)-opioids that are used in treating pain are agonists of TLR2, TLR4, other TLRs, or a combination thereof. For example, when several clinically employed (−)-opioids were tested, they were all found to be TLR4 agonists. These opioid TLR4 agonists included morphine, methadone, oxycodone, buprenorphine, fentanyl and pethadine/meperidine, amongst others.

By targeting opioid-induced activation of glial TLRs, the present inventors were able to reduce or prevent this undesirable aspect of glial activation from progressing past step A (FIG. 1) that contributes to opioid-induced tolerance, allodynia and hyperalgesia. The beneficial neuronally-induced opioid analgesia is unhindered by opioid-induced glial activation.

It is believed that at least TLR4 is responsible for initiating a component of opioid-induced glial activation that contributes significantly to the unwanted pro-nociceptive side effects of opioid administration. Accordingly, some aspects of the invention provide methods for reducing unwanted pro-nociceptive side effects of opioid administration by administering a TLR antagonist.

It has been observed that several non-selective immunosuppressive treatments ameliorate morphine withdrawal behaviors. In addition, glial involvement in pain enhancement during morphine withdrawal is blocked by IL-1 receptor antagonist or IL-10.

Co-administration of a TLR antagonist with an escalating dependence regimen of morphine significantly reduced naloxone precipitated withdrawal behaviors. Moreover, there was a corresponding reduction in glial activation in brain nuclei associated with opioid action.

In another experiment, a TLR antagonist was found to protect against previously established dependence and spontaneous withdrawal, as reflected by suppression of withdrawal induced spontaneous activity levels and weight loss. These data show that opioid-induced glial activation is involved in the development of morphine dependence and precipitation of withdrawal behaviors. Accordingly, some aspects of the invention provide methods for reducing opioid dependence, opioid withdrawal behaviors, or a combination thereof by administering a TLR antagonist. For example, the present inventors have observed that co-administration of a TLR antagonist significantly reduced withdrawal behaviors and attenuated morphine-induced weight loss.

As stated above, TLRs mediate the reinforcing and addictive actions of morphine. As such other aspects of the invention provide methods for increasing the beneficial actions, reducing the undesired effects, or a combination thereof of opioids. Such aspects of the invention often target glial activation. For example, it was observed that co-administration of a TLR antagonist resulted in a significant reduction in morphine reward.

Without being bound by any theory, it is believed that TLR-dependent glial activation results in neuropathic pain. Accordingly, some aspects of the invention provide methods for reducing neuropathic pain by modulating (e.g., reducing or preventing) TLR-dependent glial activation. One particular embodiment involves administering a TLR antagonist.

It is also believed that TLR-dependent opioid-induced glial activation results in undesired opioid side-effects, such as reducing opioid (e.g., morphine) analgesia, producing opioid dependence and reward, and causing respiratory depression. Therefore, other aspects of the invention provide methods for reducing or preventing undesired opioid side-effects, for example, reduction in opioid analgesia, dependence, reward, or a combination thereof. One particular embodiment involves administering a TLR antagonist.

The present inventors have also discovered that antagonizing TLRs or attenuating glial activation in neuropathic pain and during opioid exposure at least partially reverses allodynia and reduces unwanted opioid side effects, while maintaining opioid analgesic efficacy. The negative (i.e., undesired) side effects of opioids can be separated from the beneficial actions by, for example, targeting opioid-induced glial activation using blood brain barrier permeable pharmacotherapies such as TLR antagonists.

It is also believed that glial activation is at least partially responsible for the rewarding capacity of several abused compounds. Therefore, glial activation is a predictor for a patient's drug abuse liability. Examples of patient populations where this can pertain include HIV/AIDS, stress, and depression, etc. In all these cases, drug abuse is of considerable concern. Accordingly, some aspects of the invention provide methods for reducing or preventing drug abuse by administering a glial activation antagonist.

### Pharmaceutical Compositions

The compounds of the invention can be administered to a patient to achieve a desired physiological effect. Typically the patient is a mammal, often human. The compound can be administered in a variety of forms adapted to the chosen route of administration, i.e., orally or parenterally. Parenteral administration in this respect includes administration by the following routes: intravenous; intramuscular; subcutaneous; intraocular; intrasynovial; transepithelially including transdermal, ophthalmic, sublingual and buccal; topically including ophthalmic, dermal, ocular, rectal, inhalation (e.g., via insufflation and aerosol); intraperitoneal; rectal systemic, and central (e.g., intrathecal, such as into the cerebrospinal fluid around the spinal cord, and intracerebral into brain or CSF of the brain)

The active compound can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. Such compositions and preparation can contain at least 0.1% of active compound. The percentage of the compositions and preparation can, of course, be varied and can conveniently be between about 1 to about 10% of the weight of the unit. The amount of active compound in such therapeutically useful compositions is such that a suitable dosage will be obtained. Preferred compositions or preparations according to the present invention are prepared such that an oral dosage unit form contains from about 1 to about 1000 mg of active compound.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the active compound, sucrose as a sweetening agent, methyl and propylparabens a preservatives, a dye and flavoring such as cherry or orange flavor. Of course, any material used in preparing any dosage unit form should be pharmaceutically pure and substantially non-toxic in the amounts employed. In addition, the active compound can be incorporated into sustained-release preparations and formulation.

The active compound can also be administered parenterally. Solutions of the active compound as a free base or pharmacologically acceptable salt can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions of agents delaying absorption, e.g., aluminum monostearate and gelatin.

Sterile injectable solutions are prepared by incorporating the active compound in the required amount in the appropriate solvent with various other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

The therapeutic compounds of the invention can be administered to a mammal alone or in combination with pharmaceutically acceptable carriers, as noted above, the proportion of which is determined by the solubility and chemical nature of the compound, chosen route of administration and standard pharmaceutical practice.

The physician will determine the dosage of the present therapeutic agents which will be most suitable for prophylaxis or treatment and it will vary with the form of administration and the particular compound chosen, and also, it will vary with the particular patient under treatment. The physician will generally wish to initiate treatment with small dosages by small increments until the optimum effect under the circumstances is reached. The therapeutic dosage can generally be from about 0.1 to about 1000 mg/day, and preferably from about 10 to about 100 mg/day, or from about 0.1 to about 50 mg/Kg of body weight per day and preferably from about 0.1 to about 20 mg/Kg of body weight per day and can be administered in several different dosage units. Higher dosages, on the order of about 2× to about 4×, may be required for oral administration.

Additional objects, advantages, and novel features of this invention will become apparent to those skilled in the art upon examination of the following examples thereof, which are not intended to be limiting.

### EXAMPLES

### Methods and Materials

### General Methods

### Subjects

Pathogen-free adult male Sprague-Dawley rats (300-375 g; Harlan Labs, Madison, Wis.) were used in all experiments. Rats were housed in temperature (23±3° C.) and light (12 h:12 h light:dark cycle; lights on at 0700) controlled rooms with standard rodent chow and water available ad libitum. Each study involves n=approximately 6 per group.

### Catheter Implantation

The method of constructing and implanting the indwelling intrathecal catheters in rats was based on that described previously in Brain Research, 2000, 861, 105-116. Briefly, intrathecal catheters were implanted under anesthesia (isoflurane; Phoenix Pharmaceuticals, St. Joseph, Mo., USA) by threading sterile polyethylene-10 tubing (PE-10 Intramedic Tubing; Becton Dickinson Primary Care Diagnostics, Sparks, Md., USA) guided by an 18-gauge needle between the L5 and L6 vertebrae. The catheter was inserted 8.8 cm beyond the exterior end of the needle such that the proximal catheter tip lay over the lumbosacral enlargement. The needle was removed and the catheter was sutured to the superficial musculature of the lower back.

Catheter method 1: For acute intrathecal drug experiments the exterior end of the catheter was led subcutaneously to exit through a small incision at the nape of the neck. These catheters were preloaded with drugs at the distal end in a total volume of no greater than 25 μl. The catheters were 90 cm in length, allowing remote drug delivery without touching or otherwise disturbing the rats during the testing.

Catheter method 2: Chronic intrathecal indwelling catheters were prepared as above, but had an osmotic minipump attached to the end of the catheter. These catheters were 20 cm in total length.

Catheter method 3: For the subcutaneous catheter, a 90 cm length of PE-10 tubing was sutured to the superficial musculature of the lower back at the same time as the intrathecal catheter was implanted in these animals. The exterior end of the subcutaneous catheter paralleled the intrathecal catheter out of the same incision in the nape of the neck, allowing for remote subcutaneous administration without disturbance of the animals.

### Behavioral Measures

### Hargreaves Tests for Analgesia and Hyperalgesia

Rats received at least three 60 min habituations to the test environment prior to behavioral testing. Latencies for behavioral response to heat stimuli applied to the plantar surface of each hind-paw and tail were assessed using a modified Hargreaves test. All testing was conducted blind with respect to group assignment. Pilot studies determined that intrathecal catheter surgery did not affect baseline responses after 2 h or 7 days recovery from surgery, compared to latencies recorded prior to surgery. Briefly, baseline withdrawal values were calculated from an average of 2 consecutive withdrawal latencies of the tail and the left and the right hind-paws, measured at 15-min intervals. Latencies for the short baseline latency Hargreaves stimuli at baseline ranged from 3 to 4 s, and a cut-off time of 10 s was imposed to avoid tissue damage. Latencies for the long baseline latency Hargreaves stimuli at baseline ranged from 8 to 10 s, and a cut-off time of 20 s was imposed to avoid tissue damage. Short and long baseline latency stimuli were both employed to enable quantification of analgesia and hyperalgesia, respectively. The need for two different stimuli was due to direction of the anticipated response. Specifically, to quantify analgesia an increase in withdrawal latency was required. Therefore, short baseline responses were needed to enable 7-8 s test range before the cut-off was achieved. For hyperalgesia, a decrease in withdrawal latency needed to be quantified. Therefore, a longer baseline latency was needed to be able to detect the decrease above the bottom limit of reflex tail withdrawal. The order of paw and tail testing varied randomly. Nociceptive assessments for acute administration experiments were then made at 0 (immediately following remote drug delivery), 5 min, 15 min and every 10 min thereafter until completion of the experiment only using the short baseline latency Hargreaves stimuli. For the chronic drug delivery experiments short and long baseline latency Hargreaves stimuli were employed on alternating time points, and in these experiments the rats were tested pre dose and 2 h post dose on days 1, 4 and 7 of dosing. For twice daily injections, the nociceptive response to the morning dose was assessed.

### Von Frey Test for Mechanical Allodynia.

Rats received at least three 60 min habituations to the test environment prior to behavioral testing. Response thresholds to calibrated light pressure stimuli applied to the plantar surface of the paws was measured using the von Frey test. The test was performed using 0.406-15.136 gm calibrated Semmes-Weinstein monofilaments (von Frey hairs; Stoelting, Wood Dale, Ill., USA) as described in detail previously in Brain Research, 2000, 861, 105-116. Briefly, rats were first assessed for baseline response thresholds (average of three consecutive withdrawal assessments) from each paw at 15 min intervals, and the average response threshold from both feet was calculated. All testing was conducted blind with respect to group assignment. The behavioral responses were used to calculate the absolute threshold, by fitting a Gaussian integral psychometric function using a maximum-likelihood fitting method (see, for example, Percept.Psychophys., 1999, 61, 87-106; and Behav. Res. Methods Instrum Comput., 1986, 18, 623-632), as described in detail previously in Brain Research, 2000, 861, 105-116. Allodynia was assessed pre and post drug delivery.

### TIRAP Inhibitor Data

Rats had intrathecal catheters implanted using method 1. About 1 μL of 50 μM solution of TIRAP inhibitor or control peptide (Imgenex, San Diego, Calif.) was administered at time of intrathecal catheter implant 2 hr before administration of 15 μg of morphine. Hargreaves testing was conducted as described above. Inhibition of TRAP signaling significantly potentiated intrathecal morphine analgesia. See FIG. 5.

### LPS Antagonist Data

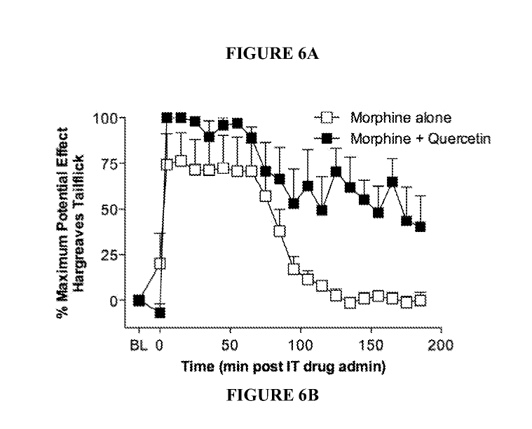

Rats had intrathecal catheters implanted using method 1. About 20 μg of lipopolysaccharide (LPS) antagonist in 4 μL (LPS from msbB E. coli mutant, which is a TLR4 antagonist due to this mutant LPS's lack of the myristoyl fatty acid moiety of lipid A, Invivogen, San Diego, Calif.) was co-administered intrathecally with morphine using a slightly modified catheter implantation protocol. Catheters were inserted only 7.7 cm rather than 8.8 cm due to the lack of diffusion within the intrathecal space by the LPS antagonist. The LPS antagonist (and therefore TLR4 antagonist) significantly potentiated morphine analgesia. See FIG. 6A.

### Potentiation of Opioid Analgesia

Rats had intrathecal catheters implanted using method 1. About 20 μg of quercetin was co-administered with morphine intrathecally resulting in a significant potentiation of analgesia. See FIG. 6B.

### Reversal of Chronic Constriction Allodynia

Following baseline Von Frey testing described above, neuropathic pain was induced using the chronic constriction injury model of partial sciatic nerve injury. CCI was performed at mid-thigh level of the left hindleg. In brief, four sterile chromic gut sutures (cuticular 4-0 chromic gut, FS-2; Ethicon, Somerville, N.J.) were loosely tied around the gently isolated sciatic nerve, in the same surgery as for intrathecal catheter placements (above). Behavioral Von Frey testing was conducted on days 4 and 10 following CCI surgery to verify the development of exaggerated pain.

### In Vitro TLR Antagonism Data

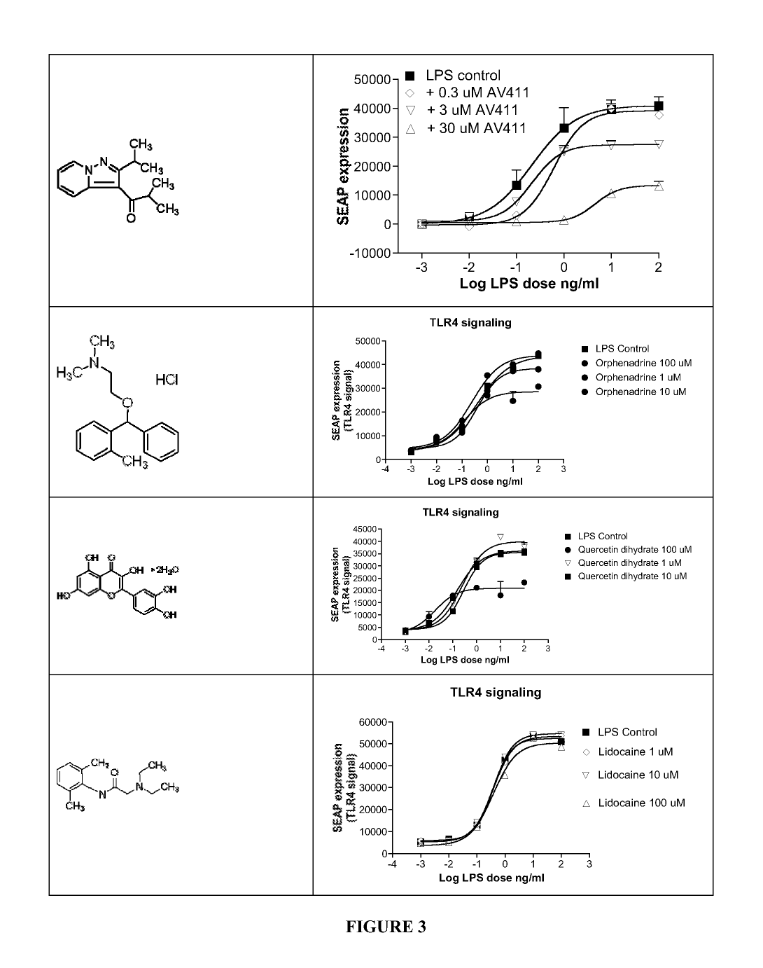

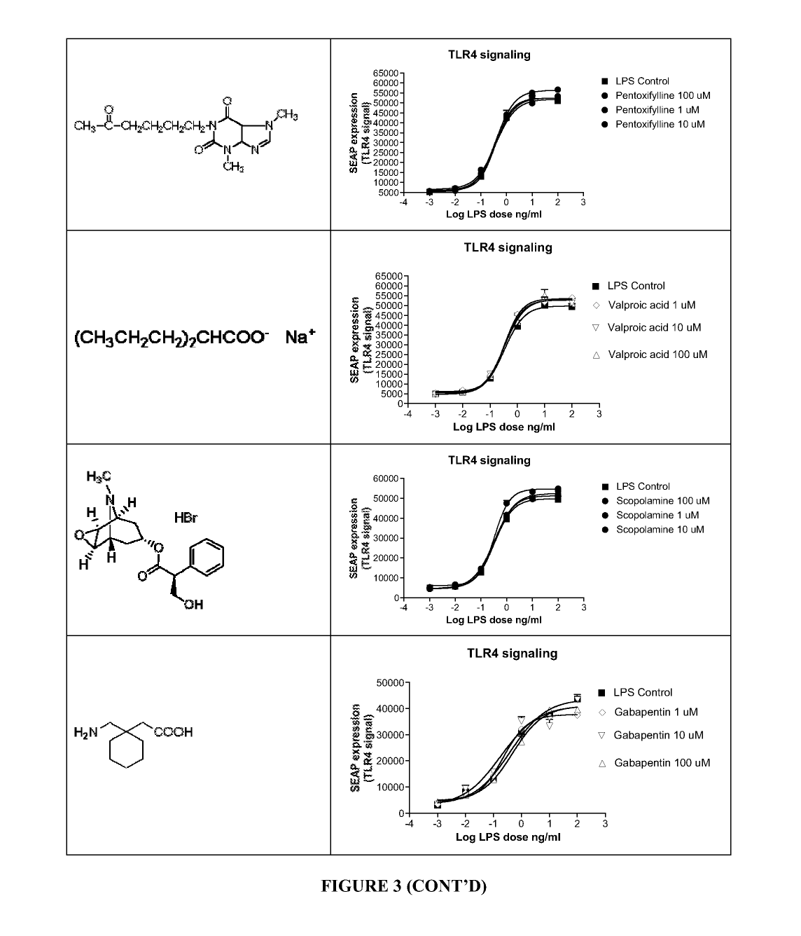

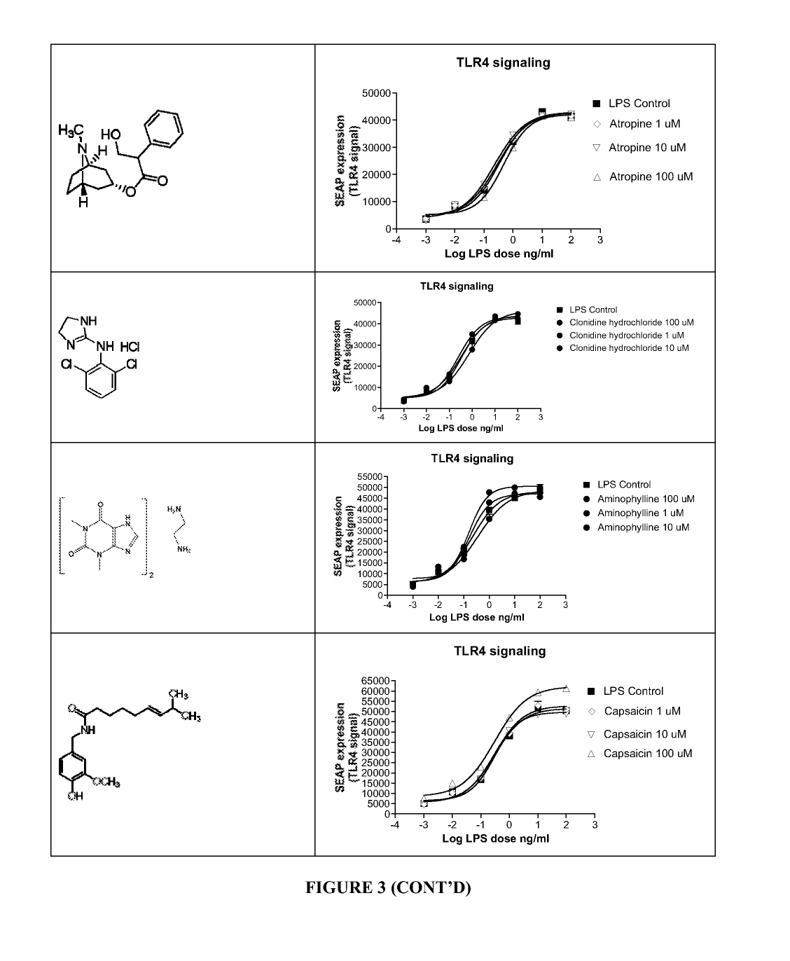

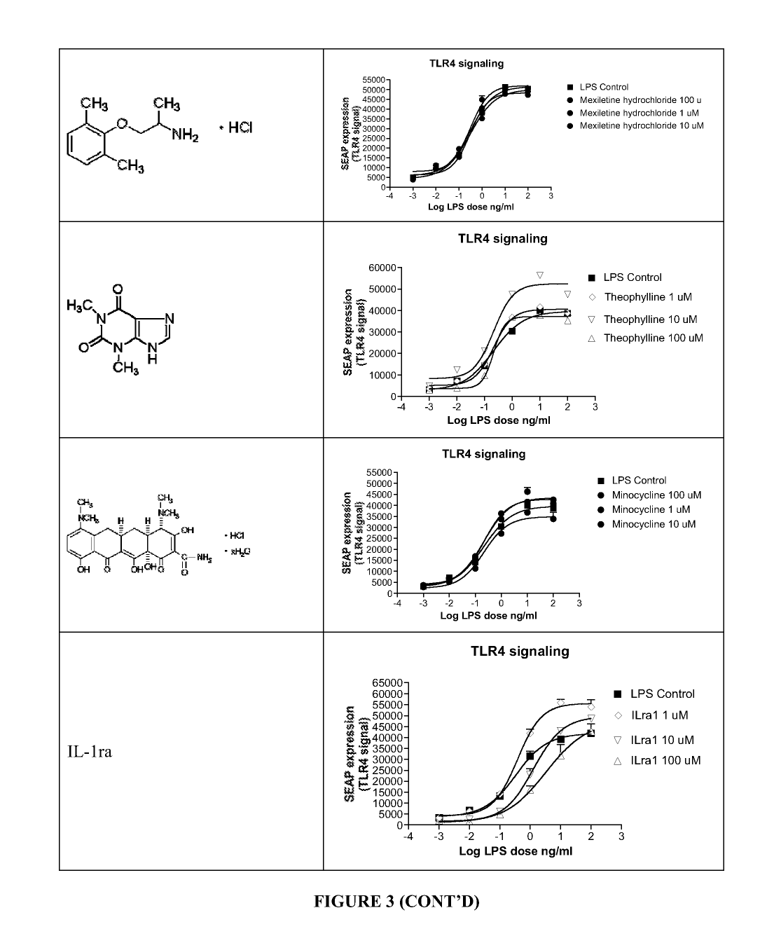

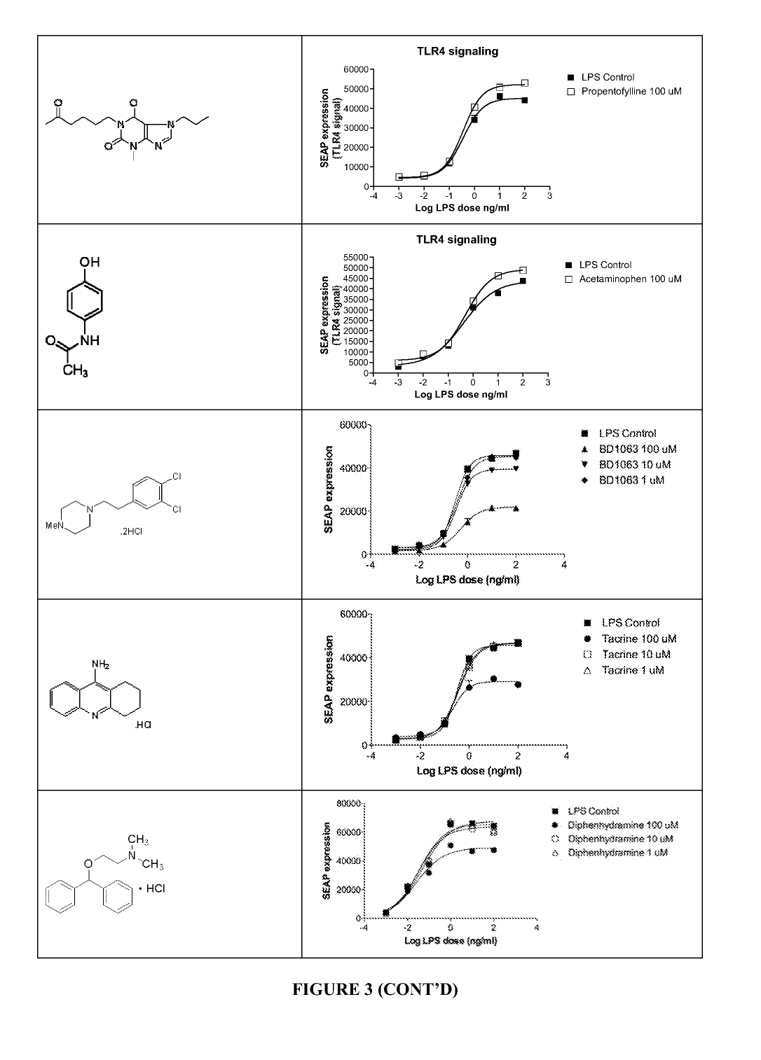

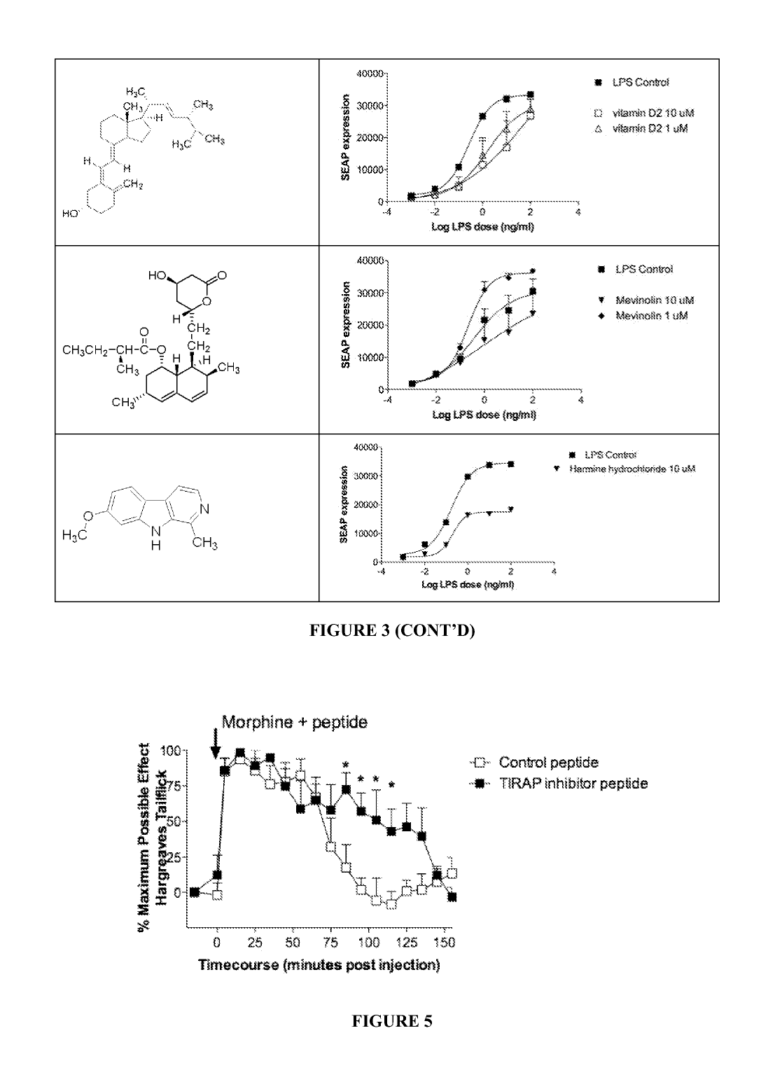

In vitro TLR antagonism data for various compounds are shown in FIG. 3. As can be seen, a wide variety of compounds antagonize TLR.

### TLR Cell Line Data

### Cell Culture and Reporter Protein Assay

A human embryonic kidney-293 (HEK 293) cell line stably transfected to express human TLR4 at high levels was purchased from Invivogen (293-htlr4a-md2cd14; here referred to as HEK-TLR4). These cells are stably transfected by Invivogen with multiple genes involved in TLR4 recognition that include TLR4 and the co-receptors MD2 and CD14. In addition, these cells stably express an optimized alkaline phosphatase reporter gene under the control of a promoter inducible by several transcription factors such as NF-kB and AP-1. Secreted alkaline phosphatase (SEAP) protein was produced as a consequence of TLR4 activation.

HEK-TLR4 cells were grown at 37° C. (5% CO₂; VWR incubator model 2300) in 10-cm dishes (Greiner Bio-One, CellStar 632171; Monroe, N.C.) in normal supplement selection media (DMEM media [Invitrogen, Carlsbad, Calif.] supplemented with 10% fetal bovine serum [Hyclone; Logan, Utah], HEK-TLR4 selection [Invivogen]; Penicillin 10,000 U/ml [Invitrogen]; Streptomycin 10 mg/ml [Invitrogen], Normocine [Invivogen], and 200 nM L-Glutamin [Invitrogen]). The cells were then plated for 48 hr in 96 well plates (Microtest 96 well plate, flat bottom, Becton Dickinson; 5×10³ cells/well) with the same media. After 48 hr, supernatants were removed and replaced with 180-μL artificial cerebrospinal fluid (sterile aCSF; 124 mM NaCl, 5 mM KCl, 0.1 mM CaCl₂ 2H₂O, 3.2 mM MgCl₂.6H₂0, 25 mM NaHCO₃, 10 mM glucose, pH 7.4) to model in vivo conditions. Drugs under test were then added in 20 μL and incubated for 24 hr. Supernatants (15 μL) were then collected from each well for immediate assay.

SEAP in the supernatants was assayed using the Phospha-Light System (Applied Biosystems) according to manufacturer's instructions. This is a chemiluminescent assay that incorporates Tropix CSPD chemiluminescent substrate. The 15 μL, test samples were diluted in 45 μL of 1× dilution buffer, transferred to 96-well plates (Thermo, Walthma, Mass.), and heated at 65° C. in a water bath (Model 210, Fisher Scientific, Pittsburgh, Pa.) for 30 min, then cooled on ice to room temperature. Assay buffer (50 μL/well) was added and, 5 min later, reaction buffer (50 μL/well) is added and allowed to incubate for 20 min at room temperature. The light output was then measured in a microplate luminometer (Dynex Technologies, #IL213.1191, Chantilly, Va.).

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed:

1. A method for treating a subject for a clinical condition associated with Toll-like receptor (TLR) activation, said method comprising administering to the subject a compound that modulates TLR-2, TLR-4, or a combination thereof.

2. The method of claim 1 further comprising administering an enantiomerically enriched (−)-opioid.

3. The method of claim 2, wherein the enantiomerically enriched (−)-opioid and the compound that modulates TLR-2, TLR-4, or a combination thereof are co-administered.

4. The method of claim 1, wherein the clinical condition associated with TLR activation comprises chronic pain, acute opioid analgesia, or a unwanted opioid side-effect, gastrointestinal pathologies, cardiovascular disease, diabetes, immune related conditions, systemic pathologies, neurodegeneration, induction of labor, fever, seizures, epilepsy, epileptogenesis, nociception, or a combination thereof.

5. The method of claim 4, wherein the clinical condition associated with TLR activation comprises chronic pain, nociception, acute opioid analgesia, or an unwanted opioid side-effect, or a combination thereof.

6. The method of claim 5, wherein the unwanted opioid side-effect comprises opioid dependence, opioid reward, opioid induced respiratory depression, opioid induced ataxia, opioid induced hyperalgesia, opioid induced allodynia or hyperalgesia, opioid induced gastrointestinal disorders, narcotic bowel syndrome, opioid dysphoria, or a combination thereof.

7. The method of claim 2, wherein the enantiomerically enriched (−)-opioid comprises morphine or a derivative or an analog thereof.

8. The method of claim 1, wherein the compound that modulates TLR-2, TLR-4, or a combination thereof comprises a compound of FIG. 3 or a derivative or an analog thereof.

9. The method of claim 1, wherein the compound is a TLR antagonist.

10. The method of claim 1, wherein the clinical condition associated with TLR activation comprises a clinical condition associated with activation of glial.

11. A method for treating neuropathic pain in a subject, said method comprising administering to the subject in need of such a treatment a TLR antagonist compound.

12. The method of claim 12 further comprising administering an enantiomerically enriched (−)-opioid.

13. The method of claim 12, wherein the enantiomerically enriched (−)-opioid and the TLR antagonist compound are co-administered.

14. The method of claim 12, wherein the enantiomerically enriched (−)-opioid is morphine or a derivative or an analog thereof.

15. An analgesic composition comprising an admixture of an enantiomerically enriched analgesic (−)-opioid and a toll-like receptor (TLR) antagonist.

16. The composition of claim 15, wherein said analgesic (−)-opioid comprises morphine or a derivative or an analog thereof.

17. The composition of claim 15, wherein said TLR antagonist antagonizes TLR-2, TLR-4, or a combination thereof.

18. The composition of claim 15, wherein said TLR antagonist comprises a compound of FIG. 3 or a derivative or an analog thereof.
